(19) United States
(12) Patent Application Publication
(10) Pub. No.: US 2026/0328923 A1
(43) Pub. Date: Oct. 22, 2026

(54) RADAR TARGET DETECTION METHOD, TERMINAL, AND COMPUTER-READABLE STORAGE MEDIUM

(71) Applicant: Possumic Technology Co., Ltd, Zhuhai (CN)

(72) Inventors: Donglin LIU, Zhuhai (CN); Hongtao LU, Zhuhai (CN); Gen ZHANG, Zhuhai (CN); Ruifeng DU, Zhuhai (CN); Jianfa ZOU, Zhuhai (CN)

(73) Assignee: Possumic Technology Co., Ltd, Zhuhai (CN)

(21) Appl. No.: 19/696,305

(22) Filed: Jun. 3, 2026

Related U.S. Application Data
(63) Continuation of application No. PCT/CN2024/141728, filed on Dec. 24, 2024.

(30) Foreign Application Priority Data
Aug. 14, 2024 (CN) 2024111133903

(51) Int. Cl. G01S 13/58; A61B 5/0205; A61B 5/024; A61B 5/05; A61B 5/08; G01S 7/35; G01S 13/42

(52) U.S. Cl. CPC G01S 13/584; A61B 5/0205; A61B 5/05; G01S 7/356; G01S 13/42; A61B 5/024; A61B 5/0816

(57) ABSTRACT

This application discloses a radar target detection method, including steps of: when a first type of target is detected, activating a second operating mode of a radar; when a second type of target is detected, activating a third operating mode of the radar; controlling the radar to extract multi-dimensional measurement parameters to detect a third type of target within a second predetermined period, and when the third type of target is detected within the second predetermined period, activating a fourth operating mode of the radar to control the radar to divide time windows to perform target detection. The solution disclosed in this application reduces the impact of micro-motion interference on the radar and lowers power consumption of the radar.

### CROSS REFERENCE TO RELATED APPLICATION

This application is a Continuation of PCT Patent Application No. PCT/CN 2024/141728 filed on Dec. 24, 2024, which claims priority of China Patent Application No. 2024111133903 filed on Aug. 14, 2024. The contents of the above-identified applications are incorporated herein by reference.

### TECHNICAL FIELD

This application relates to the field of radar technology, specifically to a radar target detection method, a terminal, and a computer-readable storage medium.

### BACKGROUND

Radar is an electronic device that detects targets using electromagnetic waves, and is widely used in various industries, such as topographic surveying, atmospheric monitoring, and autonomous driving. Target perception and decision-making using radar are widely applied in the intelligent Internet of Things (IoT). To improve the perception experience of the target, a radar device generally needs to extract multi-dimensional information of a sensed target, so as to accurately determine the presence of the target and precisely measure the target's parameters.

In actual use, the applicant has found that in scenarios with persistent interference, such as where objects in the environment are continuously blown by the wind or continuously vibrate, the radar device tends to frequently perform high computational complexity parameter measurement functions on the objects affected by environmental interference, thereby causing unnecessary power consumption of the radar device.

### SUMMARY

The embodiments of this application provide a radar target detection method, apparatus, terminal, and computer-readable storage medium that enable the radar to enter subsequent operating modes only after passing anti-interference detection, which helps reduce the impact of various micro-motion interferences in interference environments and lower the power consumption of the radar.

The embodiments of this application provide a radar target detection method, comprising:

Activating a first operating mode of a radar, and when a first type of target is detected, activating a second operating mode of the radar;

Detecting a second type of target within a first predetermined period, and when the second type of target is detected within the first predetermined period, activating a third operating mode of the radar;

Controlling the radar to extract multi-dimensional measurement parameters to detect a third type of target within a second predetermined period, and when the third type of target is detected within the second predetermined period, either activating a fourth operating mode of the radar or controlling the radar to remain in the third operating mode, wherein the multi-dimensional measurement parameters include range, velocity, angle, respiration and heartbeat rate, micro-Doppler feature, and motion trajectory; and

In the fourth operating mode, controlling the radar to divide time windows to perform target detection, and when a desired target is detected within one time window, controlling the radar to proceed to a next time window to continue detecting the presence of the desired target, wherein the desired target includes the first type of target, the second type of target, and the third type of target, a set of the first type of target includes the second type of target, and a set of the second type of target includes the third type of target.

In some embodiments, after activating the first operating mode of the radar, the method further comprises:

Periodically transmitting a first frame format detection signal and performing a first type of target detection based on a received signal;

When the radar is a ranging radar, generating a range spectrum based on the received signal of each frame and performing the first type of target detection on the range spectrum; and

When the radar is a Doppler radar, generating a velocity spectrum based on the received signal of each frame and performing the first type of target detection on the velocity spectrum.

In some embodiments, after activating the second operating mode of the radar, the method further comprises:

Transmitting a second frame format detection signal for a plurality of times, and weight-combining data with a same sampling point number among all received signals to obtain a combined signal, wherein transmitted second frame format detection signals are arranged at unequal time intervals;

When the radar is a ranging radar, generating a range spectrum based on the combined signal and performing a second type of target detection on the range spectrum; and

When the radar is a Doppler radar, computing an energy value of the combined signal and performing the second type of target detection based on a comparison result between the energy value and an energy threshold.

In some embodiments, after activating the second operating mode of the radar, the method further comprises:

Transmitting a second frame format detection signal for a plurality of times and performing forward-backward difference processing on all received signals in order of reception to obtain a plurality of differential signals, wherein transmitted second frame format detection signals are arranged at unequal time intervals;

When the radar is a ranging radar, generating a plurality of range spectra based on the plurality of differential signals and performing a second type of target detection based on a number of detected targets in the plurality of range spectra; and

When the radar is a Doppler radar, computing energy values of the plurality of differential signals and performing the second type of target detection based on a number of the energy values that exceed an energy threshold.

In some embodiments, controlling the radar to extract multi-dimensional measurement parameters comprises:

Periodically transmitting a third frame format detection signal and receiving corresponding echo signals;

When the radar is a ranging radar, generating a range-velocity spectrum based on a plurality of received echo signals for a same frame and obtaining the multi-dimensional measurement parameters based on a plurality of range-velocity spectra; and

When the radar is a Doppler radar, generating a velocity spectrum based on a plurality of received echo signals for a same frame and obtaining the multi-dimensional measurement parameters based on a plurality of velocity spectra.

In some embodiments, controlling the radar to divide the time windows to perform the target detection comprises:

Performing joint processing on all echo signals received within a current time window to perform the target detection, wherein performing the joint processing is selected from one of:

Performing a two-dimensional Fourier transform on all the echo signals to obtain a range-velocity spectrum;

Performing arithmetic averaging on the data with a same sampling point number among all the echo signals and computing a range spectrum based on arithmetically averaged data;

Weight-combining the data with the same sampling point number among all the echo signals to obtain a combined signal and generating a range spectrum based on the combined signal; and

Performing forward-backward difference processing on all the echo signals in order of reception to obtain a plurality of differential signals, and respectively calculating a range spectrum for each of the plurality of differential signals to obtain a plurality of range spectra.

In some embodiments, after detecting a target in the second and fourth operating modes, the method further comprises:

Obtaining angle information of a detected target, wherein the angle information includes a horizontal angle, an azimuth angle, and an elevation angle;

Determining whether the angle information is within a predetermined range;

Determining that the detected target is the desired target under a current operating mode when the angle information is within the predetermined range;

Determining that the detected target is not the desired target under the current operating mode when the angle information is not within the predetermined range; and

Wherein the desired target is the second type of target in the second operating mode, and the desired target includes the first type of target, the second type of target, and the third type of target in the fourth operating mode.

The embodiments of this application also provide a radar target detection apparatus, comprising:

A first detection unit, configured to activate a first operating mode of a radar, and when a first type of target is detected, activate a second operating mode of the radar;

A second detection unit, configured to detect a second type of target within a first predetermined period, and when the second type of target is detected within the first predetermined period, activate a third operating mode of the radar;

A third detection unit, configured to control the radar to extract multi-dimensional measurement parameters to detect a third type of target within a second predetermined period, and when the third type of target is detected within the second predetermined period, either activate a fourth operating mode of the radar or control the radar to remain in the third operating mode, wherein the multi-dimensional measurement parameters include range, velocity, angle, respiration and heartbeat rate, micro-Doppler feature, and motion trajectory; and

A fourth detection unit, configured to, in the fourth operating mode, control the radar to divide time windows to perform target detection, and when a desired target is detected within one time window, control the radar to proceed to a next time window to continue detecting the presence of the desired target, wherein the desired target includes the first type of target, the second type of target, and the third type of target, a set of the first type of target includes the second type of target, and a set of the second type of target includes the third type of target.

The embodiments of this application also provide a terminal, comprising: a memory and a processor, wherein the memory stores an application processing program, and when executed by the processor, the application processing program implements the steps of any radar target detection method provided in the embodiments of this application.

The embodiments of this application also provide a computer-readable storage medium that stores a plurality of instructions, wherein the plurality of instructions are executed by a processor to perform any radar target detection method provided in the embodiments of this application.

The radar target detection method provided in the embodiments of this application can activate the first operating mode of the radar, activate the second operating mode of the radar when the first type of target is detected, detect the second type of target within the first predetermined period, activate the third operating mode of the radar when the second type of target is detected, control the radar to extract multi-dimensional measurement parameters and detect the third type of target within the second predetermined period, activate the fourth operating mode of the radar or control the radar to remain in the third operating mode when the third type of target is detected within the second predetermined period, control the radar to divide time windows to perform target detection in the fourth operating mode, and control the radar to proceed to the next time window to continue detecting the presence of the desired target if the desired target is detected within one time window, wherein the desired target includes the first, second, and third types of targets, the set of the first type of target includes the second type of target, and the set of the second type of target includes the third type of target. The solution provided in the embodiments of this application can selectively activate the next operating mode of the radar based on the target detection results of the previous operating mode, gradually activating the multi-dimensional parameter measurement function of the radar, which helps reduce the impact of various micro-motion interferences in interference environments and lower the power consumption of the radar.

### BRIEF DESCRIPTION OF THE DRAWINGS

To more clearly illustrate the technical solutions in the embodiments of this application, the accompanying drawings required for describing the embodiments will be briefly introduced below. It is obvious that the accompanying drawings described hereinafter only cover some embodiments of this application, and those skilled in the art can derive other accompanying drawings from these drawings without creative work.

FIG. 1 illustrates a schematic flowchart of a radar target detection method provided in an embodiment of this application;

FIG. 2 illustrates a schematic structural diagram of a radar target detection apparatus provided in an embodiment of this application;

FIG. 3 illustrates a schematic structural diagram of a terminal provided in an embodiment of this application.

### DETAILED DESCRIPTION OF THE EMBODIMENTS

Herein, exemplary embodiments will be described in detail with reference to the examples shown in the accompanying drawings. When the following description refers to the accompanying drawings, unless otherwise specified, the same reference numerals in different drawings represent the same or similar elements. The implementation methods described in the following exemplary embodiments do not cover all implementation methods consistent with this application. Instead, they are merely examples of apparatuses and methods consistent with some aspects of this application as detailed in the appended claims.

It should be noted that, in this application, the terms “include,” “comprise,” or any of their variants are intended to cover non-exclusive inclusions. Specifically, a process, method, article, or apparatus that includes a series of elements shall include not only those elements but also other elements that are not explicitly listed or inherently associated with such process, method, article, or apparatus. Without additional limitations, an element defined by the phrase “including one . . . ” shall not exclude the presence of additional identical elements in the process, method, article, or apparatus that includes the element. Furthermore, components, features, or elements with the same names in different embodiments of this application may have the same or different connotations, and their specific meanings shall be determined based on their interpretation in the specific embodiment or further in conjunction with the context of that embodiment.

It should be understood that although the steps in the flowcharts of the embodiments of this application are shown sequentially by arrow indications, these steps are not necessarily executed in the sequence indicated by the arrows. Unless explicitly stated otherwise in this application, there is no strict sequential restriction on the execution of these steps, and they may be executed in other sequences. Furthermore, at least some of the steps in the drawings may include multiple sub-steps or stages, which are not necessarily completed simultaneously but may be executed at different times. The order of execution is not necessarily sequential; instead, it may be performed alternately or in rotation with at least a part of other steps and their sub-steps or stages.

It should be noted that in this application, step codes such as 101 and 102 are used to express the corresponding content more clearly and concisely, and do not constitute substantive limitations on the order of the steps. Those skilled in the art may perform step 102 prior to step 101 in specific implementation, and all such variations shall fall within the protection scope of this application.

The reference to “embodiment” in this application means that specific features, structures, or characteristics described in conjunction with the embodiment may be included in at least one embodiment of this application. The appearances of this phrase at various positions in the specification do not necessarily refer to the same embodiment, nor independent or alternative embodiments that are mutually exclusive of other embodiments. Those skilled in the art will explicitly and implicitly understand that the embodiments described in this application may be combined with other embodiments.

As used herein, the phrases “presence of a/the first type of target,” “a/the first type of target is detected,” and “a/the first type of target has not disappeared” carry the same meaning; the phrases “absence of a/the first type of target,” “no first type of target is detected,” and “a/the first type of target has disappeared” carry the same meaning; the phrases “presence of a/the second type of target,” “a/the second type of target is detected,” and “a/the second type of target has not disappeared” carry the same meaning; the phrases “absence of a/the second type of target,” “no second type of target is detected,” and “a/the second type of target has disappeared” carry the same meaning; the phrases “presence of a/the third type of target,” “a/the third type of target is detected,” and “a/the third type of target has not disappeared” carry the same meaning; the phrases “absence of a/the third type of target,” “no third type of target is detected,” and “a/the third type of target has disappeared” carry the same meaning.

Embodiments of this application provide a radar target detection method. The method may be performed by a radar target detection apparatus provided in the embodiments herein, or by an intelligent terminal or a server integrated with such an apparatus. The radar target detection apparatus may be implemented in hardware or software.

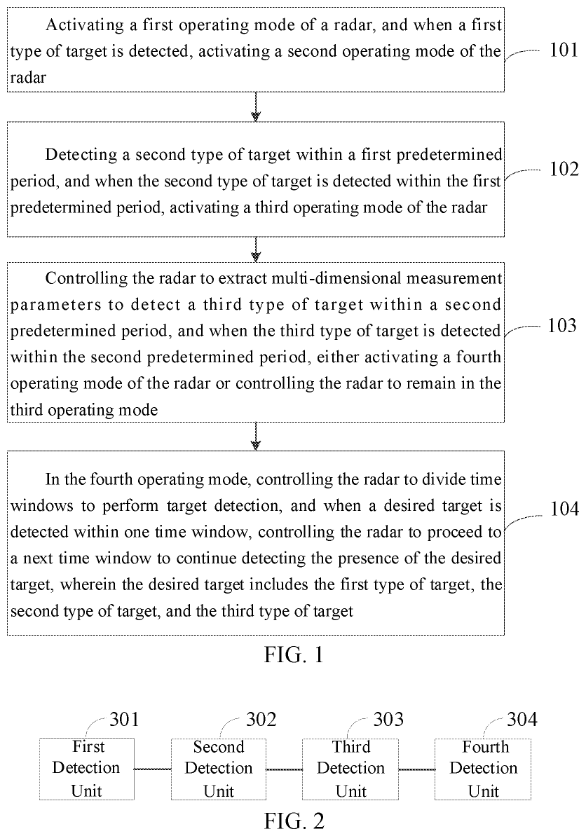

Referring to FIG. 1, a schematic flowchart of a radar target detection method provided in an embodiment of this application is illustrated. Detailed steps of the radar target detection method may be as follows:

101, activating a first operating mode of a radar, and when a first type of target is detected, activating a second operating mode of the radar.

Specifically, after being powered on, the radar enters the first operating mode. In this state, the radar periodically transmits a first frame format detection signal and detects the first type of target frame by frame. When the first type of target is detected, the radar switches to the second operating mode; otherwise, the radar remains in the first operating mode.

In some embodiments, when the radar is a Frequency Modulated Continuous Wave (FMCW) radar or a Stepped Frequency Continuous Waveform (SFCW) radar, the first frame format detection signal of each frame may be configured to include a ranging signal waveform. For an FMCW radar, the ranging signal waveform corresponds to a chirp signal, whereas for an SFCW radar, the ranging signal waveform corresponds to an SFCW waveform including a plurality of stepped frequency points. In the following descriptions of this application, the ranging signal waveform has the above meaning. After receiving and sampling the received signal of each frame, and suppressing stationary components in a slow-time dimension, the radar computes and generates a range spectrum. The radar detects targets from the generated range spectrum and filters detected targets according to a predetermined range threshold. If one or more targets exist within the predetermined range threshold, the presence of the first type of target is determined; otherwise, the absence thereof is determined. Wherein, the steps of suppressing stationary components in the slow-time dimension, generating a range spectrum, and detecting targets on the range spectrum may be implemented using conventional techniques in the art, and this application imposes no limitation on the specific implementation thereof. In the embodiments, the first type of target refers to non-stationary targets filtered by the range threshold subsequent to stationary component suppression.

In some embodiments, when the radar is a Doppler radar or an SFCW radar, the first frame format detection signal of each frame may be configured as a Doppler radar signal waveform. For the SFCW radar, the Doppler radar signal waveform refers to an SFCW waveform including only one frequency point, such that the SFCW radar operates equivalently to a Doppler radar. In the following descriptions of this application, the Doppler radar signal waveform of an SFCW radar has the above meaning. After receiving and sampling the received signal of each frame, the radar feeds the sampled signal into a high-pass filter and computes an energy value of an output signal of the high-pass filter. When the energy value exceeds a predetermined energy threshold, the presence of the first type of target is determined; otherwise, the absence thereof is determined. Herein, the first type of target refers to non-stationary targets filtered by the energy threshold subsequent to stationary component suppression by the high-pass filter. It will be appreciated that, in this embodiment, by utilizing the correlation between radar echo energy intensity and range, indirect range filtering is performed through the predetermined energy threshold.

In some embodiments, when the radar is a Doppler radar or an SFCW radar, the first frame format detection signal of each frame may be configured as a plurality of Doppler radar signal waveforms arranged at equal time intervals. After receiving and sampling the received signal of each frame and suppressing stationary components in the slow-time dimension thereof, the radar computes and generates a velocity spectrum. The radar detects targets from the generated velocity spectrum and filters the detected targets based on a predetermined velocity threshold. If one or more targets still fall within a predetermined velocity threshold range, the presence of the first type of target is determined; otherwise, the absence thereof is determined. Wherein, the steps of suppressing stationary components in the slow-time dimension, generating a velocity spectrum, and detecting targets on the velocity spectrum may be implemented using conventional techniques in the art, and this application imposes no limitation on the specific implementation thereof. Herein, the first type of target refers to non-stationary targets filtered by the velocity threshold subsequent to stationary component suppression.

It will be appreciated that, in the embodiments of this application, the ranging radar refers to an FMCW radar or an SFCW radar that transmits a plurality of stepped frequency points. In the above embodiments, when the radar operates in the first operating mode, it simply and efficiently detects the presence of the first type of target only from the range dimension or Doppler (velocity) dimension. However, it cannot effectively distinguish whether the first type of target constitutes micro-motion interference in the environment, such as swaying of flowers, plants, or trees, or vibration of objects in a typical household environment. When the first type of target is detected in the first operating mode, the radar switches to the second operating mode to detect and confirm the presence of a second type of target. In the first operating mode, to save the radar's power consumption, the frame rate at which the radar transmits the first frame format detection signal may be set to 0.5 Hz, 1 Hz, 2 Hz, 3 Hz, 4 Hz, or 5 Hz, depending on detection environments and response latency requirements of the radar application. This application imposes no limitation on this frame rate.

102, detecting a second type of target within a first predetermined period, and when the second type of target is detected within the first predetermined period, activating a third operating mode of the radar.

Specifically, the radar switches to the second operating mode to perform further target confirmation and interference rejection so as to confirm the presence of the second type of target. Specifically, when one or more second type of targets are detected within T₀ time, the radar switches to the third operating mode; when no second type of target is detected within T₀ time, the radar switches back to the first operating mode. Wherein, T₀ is a first predetermined period, representing a maximum time the radar remains in the second operating mode. It may be set to 0.5 s, 1 s, or 2 s, depending on detection environments and response latency requirements of the radar application. This application does not impose any limitation on the first predetermined period T₀.

When the radar is in the second operating mode, the radar transmits the second frame format detection signal a total of N times. A time interval between the nth transmission of detection signal and the (n−1)th transmission of detection signal is denoted as tₙ, wherein n=2, 3, . . . , N, and N may be 2, 3, 4, or 5. Values of the time interval tₙ are not all identical. The time intervals tₙ (n=2, 3, . . . , N) satisfy t₂+t₃+. . . +t_{N}<T₀. The sequence of tₙ, n=2,3, . . . , N may be preset based on non-uniform sampling methods or sparse sequence design methods, combined with Doppler features of micro-motion interference in the environment.

In some embodiments, the radar is an FMCW radar or an SFCW radar. The second frame format detection signal of each frame is configured as the ranging signal waveform. It will be appreciated that, in the second operating mode, N frames of the second frame format detection signal are cumulatively transmitted. After receiving and sampling the nth frame received signal, the radar obtains the nth frame echo sampled signal, expressed as r(n)=[r(n,1), r(n,2), . . . , r(n,h), . . . , r(n,H)], wherein n=1, 2, 3, . . . , N, h=1, 2, 3, . . . , H, wherein H represents the number of sampling points in the echo sampled signal of each frame, and r(n,h) represents the hth sampling point of the echo sampled signal of the nth frame. The radar performs weighted combination on data with a same sampling point number in all the received signals in the slow-time dimension to obtain a combined signal. Specifically, the combined signal is represented as rw=[rw(1), rw(2), . . . , rw(h), . . . , rw(H)], wherein rw(h) is the hth element of the combined signal, rw(h)=x(h)*w, x(h)=[r(1,h), r(2,h), . . . , r(n,h), . . . , r(N,h)] is a row vector, and w is a preset N*1 weighted column vector with high-pass characteristics. The radar uses the combined signal to generate the range spectrum, detects targets from the generated range spectrum, and filters the detected targets according to the predetermined range threshold. When there are one or more targets within the predetermined range threshold, it is determined that the second type of target is present; otherwise, it is determined that the second type of target is absent. The steps of generating the range spectrum and detecting the targets on the range spectrum may be implemented using conventional techniques in the art, and this application imposes no limitation on the detection details. Herein, the second type of target refers to targets detected by filtering with the predetermined range threshold after weighted combination suppression of low-speed interference.

In some embodiments, the radar is a Doppler radar or an SFCW radar. The second frame format detection signal of each frame is configured to include one Doppler radar signal waveform. It will be appreciated that, in the second operating mode, N frames of the second frame format detection signal are cumulatively transmitted. After receiving and sampling the nth frame received signal, the radar obtains the nth frame echo sampled signal, denoted as r(n), wherein n=1, 2, 3, . . . , N. The radar performs weighted combination on data with a same sampling point number in all the received signals in the slow-time dimension to obtain a combined signal. Specifically, the combined signal is expressed as rw=x*w, wherein x=[r(1), r(2), . . . , r(n), . . . , r(N)] is a row vector, and w is a preset N*1 weighted column vector with high-pass characteristics. The radar computes an energy value of the combined signal. When the energy value exceeds a predetermined energy threshold E, one or more second type of targets are detected; otherwise, no second type of target is detected. Herein, the second type of target refers to targets filtered by the predetermined energy threshold after weighted combination suppression of low-speed interference. It will be appreciated that, in this embodiment, by utilizing the correlation between the radar echo energy intensity and range, indirect range filtering is performed through the predetermined energy threshold.

In some embodiments, the radar is an FMCW radar or an SFCW radar. The second frame format detection signal of each frame is configured as the ranging signal waveform. It will be appreciated that, in the second operating mode, N frames of the second frame format detection signal are cumulatively transmitted. After receiving and sampling the received signal of the nth frame, the radar obtains the nth frame echo sampled signal, denoted as r(n)=[r(n,1), r(n,2), . . . , r(n, h), . . . , r(n,H)], wherein n=1, 2, 3, . . . , N, h=1, 2, 3, . . . , H, wherein H denotes the number of sampling points in the echo sampled signal of each frame, and r(n,h) represents the hth sampling point of the echo sampled signal of the nth frame. The radar performs forward-backward difference processing on the N received signals in the slow-time dimension in the order of reception to obtain N−1 differential signals. Specifically, the lth differential signal is expressed as rd(l)=[r(l+1,1)−r(l,1), r(l+1,2)−r(l+l,2), . . . , r(l+1,h)−r(l, h), . . . , r(l+1,H)−r(l, H)], wherein l=1, 2, 3, . . . , N−1, and r(l,h) denotes the hth sampling point of the lth frame echo sampled signal. The radar generates N−1 range spectra from the N−1 differential signals, performs target detection from the generated range spectra, and filters the detected targets in each range spectrum according to a predetermined range threshold. If one or more targets within the predetermined range threshold are detected in K of the N−1 range spectra, and K is greater than a predetermined threshold value K0, it is determined that a second type of target is detected; otherwise, it is determined that there is no second type of target. K0 may be set to 1, 2, 3, or 4, and does not exceed N−1. The steps of generating the range spectrum and detecting the targets on the range spectrum may be implemented using conventional techniques in the art, and this application imposes no limitation on the detection method and details thereof. Herein, the second type of target refers to targets filtered by the predetermined range threshold after differential suppression of low-speed interference.

In some embodiments, the radar is a Doppler radar or an SFCW radar. The second frame format detection signal of each frame is configured to include one Doppler radar signal waveform. It will be appreciated that, in the second operating mode, N frames of the second frame format detection signal are cumulatively transmitted. After receiving and sampling the nth frame received signal, the radar obtains an echo sampled signal of the nth frame, denoted as r(n), wherein n=1, 2, 3, . . . , N. The radar performs forward-backward difference processing on the N received signals in the slow-time dimension in the order of reception to obtain N−1 differential signals. Specifically, the lth differential signal is expressed as rd(l)=r(l+1)−r(l), wherein l=1, 2, 3, . . . , N−1. The radar computes the energy values of the N−1 differential signals. When K energy values among the N−1 energy values are greater than a predetermined energy threshold, and K is greater than the predetermined threshold value K0, it is determined that the second type of target is detected; otherwise, it is determined that there is no second type of target. K0 may be set to values of 1, 2, 3, or 4, and the value of K0 does not exceed N−1. Herein, the second type of target refers to targets filtered by the predetermined energy threshold after differential suppression of low-speed interference. It will be appreciated that, in this embodiment, by utilizing the correlation between the radar echo energy intensity and range, indirect range filtering is performed through the predetermined energy threshold.

When the radar operates in the second operating mode and supports angle measurement, the angle measurement may be enabled to obtain angle information, which is used to further confirm whether the targets detected after range or velocity filtering in each embodiment of the above-mentioned second operating mode are within a predetermined angle range. The angle information includes a horizontal angle, an azimuth angle, and an elevation angle. When the targets detected after range or velocity filtering in the second operating mode are within the predetermined angle range (i.e., predetermined horizontal angle, predetermined azimuth angle, and predetermined elevation angle range), it is determined that the detected target is the desired target in the second operating mode (i.e., the second type of target); otherwise, the detected target is not the second type of target. When, in the second operating mode, none of the targets detected after range or velocity filtering is within the predetermined angle range, it is determined that there is no second type of target; otherwise, it is determined that there is the second type of target.

It will be appreciated that, when the radar operates in the second operating mode, the N frames of the second frame format detection signal are configured with unequal time intervals. Compared with equal time intervals, high-pass filtering can be implemented in the slow-time dimension with a relatively small value of N, which helps reduce the radar's power consumption in the second operating mode while suppressing micro-motion interference in the environment. That is, on the premise that the first type of target has been detected in the first operating mode, the impact of micro-motion interference in the environment is further eliminated. When the targets are still detected, it is determined that there is a second type of target, and the radar may switch to the third operating mode.

103, controlling the radar to extract multi-dimensional measurement parameters to detect a third type of target within a second predetermined period, and when the third type of target is detected within the second predetermined period, either activating a fourth operating mode of the radar or controlling the radar to remain in the third operating mode.

Wherein the multi-dimensional measurement parameters described above include range, velocity, angle, respiration and heartbeat rate, micro-Doppler feature, and motion trajectory. Specifically, when the radar switches to the third operating mode, the radar performs accurate extraction and tracking of multi-dimensional measurement parameters, including range, velocity, angle, respiration and heartbeat rate, micro-Doppler feature, and motion trajectory. The second predetermined period is denoted as T₁. When no third type of target is detected within the second predetermined period T₁, the radar switches back to the first operating mode. When the third type of target is detected within the second predetermined period T₁, the radar selectively switches to the fourth operating mode to determine whether the desired target has disappeared in the fourth operating mode; or the radar selectively continues to remain in the third operating mode to determine whether the third type of target has disappeared. That is, if no third type of target is detected within a continuous period T₁, it is determined that the third type of target has disappeared, and the radar switches back to the first operating mode.

When operating in the third operating mode, the radar transmits a frame of third frame format detection signal every Δt₀ and receives corresponding echo signals. For a ranging radar, the third frame format detection signal of each frame contains Q ranging signal waveforms arranged at equal time intervals. The radar generates a range-velocity spectrum using Q received echo signals corresponding to the same frame and further extracts the measurement parameters of the targets from the plurality of range-velocity spectra. Herein, Δt₀ is greater than or equal to a product of Q and Δt₁, and Δt₀, Δt₁, and Q are preset values. Δt₁ represents a time interval between the Q signal waveforms arranged at equal time intervals in the same frame. For a Doppler radar, the third frame format detection signal of each frame includes Q Doppler radar signal waveforms arranged at equal time intervals. The radar generates a velocity spectrum using the Q received echo signals corresponding to the same frame and further extracts the measurement parameters of the targets from the plurality of velocity spectra. Herein, Δt₀ is greater than or equal to a product of Q and Δt₁, and Δt₀, Δt₁, and Q are preset values. Δt₁ represents a time interval between the Q signal waveforms arranged at equal time intervals in the same frame.

In some embodiments, the radar is an FMCW radar or an SFCW radar, configured to detect human movement as the third type of target. The radar transmits a frame of the third frame format detection signal every Δt₀ and receives corresponding echo signals. The third frame format detection signal of each frame contains Q ranging signal waveforms arranged at equal time intervals. After performing static component suppression, the radar generates a range-velocity spectrum using the Q received echo signals corresponding to the same frame. It further extracts the target's measurement parameters, including range, velocity, angle, micro-Doppler feature, and motion trajectory from a plurality of obtained range-velocity spectra. Based on the plurality of obtained range-velocity spectra, the presence of the third type of target may be determined using methods such as kinematic model point cloud data association, Kalman filter tracking, cluster analysis, or deep learning. In this mode, when the radar is a MIMO radar, all transmitting and receiving antennas of the radar form an antenna array and participate in parameter measurement and extraction. The specific detection method for human movement based on the ranging radar (FMCW radar or SFCW radar) may be implemented using conventional techniques in the art, and this application imposes no limitation on the specific detection method for the third type of target (human movement).

When the third type of target is not detected within the second predetermined period T₁, the radar switches back to the first operating mode. When the third type of target is detected within the second predetermined period T₁, the radar selectively switches to the fourth operating mode, or continues to remain in the third operating mode and perform a disappearance judgment on the third type of target. That is, when the third type of target is not detected within a continuous period T₁, it is determined that the third type of target has disappeared, and the radar switches back to the first operating mode. The second predetermined period T₁ is set as an integer multiple of Δt₀.

In some embodiments, the radar is an FMCW radar or an SFCW radar, configured to detect human vital signs as the third type of target. The radar transmits a frame of the third frame format detection signal every Δt₀ and receives corresponding echo signals. The third frame format detection signal of each frame includes Q ranging signal waveforms. After static component suppression, the radar generates a range-velocity spectrum using the Q received echo signals corresponding to the same frame. It further extracts the target's measurement parameters, including range, velocity, angle, micro-Doppler feature, and motion trajectory, from a plurality of obtained range-velocity spectra. Based on the plurality of obtained range-velocity spectra, human respiratory and heartbeat characteristics are extracted using methods such as kinematic model point cloud data association based on human vital signs, cluster analysis, or deep learning, thereby determining the presence of the third type of target. In this mode, when the radar is a MIMO radar, all transmitting and receiving antennas of the radar form an antenna array and participate in parameter measurement and extraction. The specific detection method for human vital signs based on the ranging radar (FMCW radar or SFCW radar) may be implemented using conventional techniques in the art, and this application imposes no limitation on the specific detection method for the third type of target (human vital signs).

When the third type of target is not detected within the second predetermined period T₁, the radar switches back to the first operating mode. When the third type of target is detected within the second predetermined period T₁, the radar may selectively switch to the fourth operating mode, or continue to remain in the third operating mode and perform a disappearance judgment on the third type of target. That is, when the third type of target is not detected within a continuous period T₁, it is determined that the third type of target has disappeared, and the radar switches back to the first operating mode. T₁ is set as an integer multiple of Δt₀.

In some embodiments, the radar is a Doppler radar, configured to detect human vital signs as the third type of target. The radar transmits a frame of the third frame format detection signal every Δt₀ and receives corresponding echo signals. The third frame format detection signal of each frame includes Q Doppler radar signal waveforms arranged at equal time intervals. The radar generates a velocity spectrum using the Q received echo signals corresponding to a same frame and further extracts the target's measurement parameters, including velocity, angle, micro-Doppler feature, etc., from a plurality of obtained velocity spectra. Based on the plurality of obtained velocity spectra, human respiratory and heartbeat characteristics are extracted using methods such as kinematic model point cloud data association based on human vital signs, cluster analysis, or deep learning, thereby determining the presence of the third type of target. In this mode, when the radar is a MIMO radar, all transmitting and receiving antennas of the radar form an antenna array and participate in parameter measurement and extraction. The specific detection method for human vital signs based on Doppler radar may be implemented using conventional techniques in the art and this application does not impose limitations on the specific detection method for the third type of target (human vital signs). When the third type of target is not detected within the second predetermined period T₁, the radar switches back to the first operating mode. When the third type of target is detected within the second predetermined period T₁, optionally, the radar may selectively switch to the fourth operating mode or continue to remain in the third operating mode, and perform a disappearance judgment on the third type of target. That is, when the third type of target is not detected within a continuous period T₁, it is determined that the third type of target has disappeared, and the radar switches back to the first operating mode. T₁ is set as an integer multiple of Δt₀.

When operating in the third operating mode, the radar performs target parameter measurement and target detection with high computational load. Entering the third operating mode only after determining the potential presence of the third type of target in the first and second operating modes helps to avoid frequent operation of parameter measurement and detection algorithms with high computational load in interference environments, thereby reducing the radar's power consumption. When detecting the third type of target in the third operating mode, the radar sends an indication signal indicative of the third type of target detection to a main control unit, which then performs subsequent linked operations, such as sending instructions to the controlled device via a wired or wireless connection to request a response action. The manner in which the radar detects a target, notifies the main control unit upon detection, and enables the main control unit to control linked operations of the controlled device is well-known in the art. This application imposes no limitation on such related implementations.

104, in the fourth operating mode, controlling the radar to divide time windows to perform target detection, and when a desired target is detected within one time window, controlling the radar to proceed to a next time window to continue detecting the presence of the desired target, wherein the desired target includes the first type of target, the second type of target, and the third type of target.

Specifically, when switching to the fourth operating mode, the radar transmits a fourth frame format detection signal to perform a disappearance judgment on the target. That is, if no target is found within the current time window, the radar switches back to the first operating mode. If a target is found within the current time window, the radar proceeds to the next time window to continue detecting whether the target has disappeared. Each time window has the same duration. In the fourth operating mode, the desired target includes the first type of target, the second type of target, and the third type of target. The set of the first type of target includes the second type of target, and the set of the second type of target includes the third type of target. The desired target in the fourth operating mode may be pre-selected from the three types of targets based on the radar's application scenario and system power consumption design. The radar performs joint processing on all received signals within each time window to confirm whether the target has disappeared in the current time window.

In the fourth operating mode, joint processing using all received signals within each time window may provide superior signal-to-noise ratio (SNR) gain. The joint processing operations include:

Joint processing operation 1: performing a 2D discrete Fourier transform on all received echo signals within one time window to obtain one range-velocity spectrum.

Joint processing operation 2: calculating the arithmetic mean of data with the same sampling point number from all the received echo signals within one time window and computing one range spectrum from the arithmetically averaged data.

Joint processing operation 3: performing weighted combination on data with the same sampling point number from all the received echo signals within one time window to obtain a combined signal and generating one range spectrum using the combined signal.

Joint processing operation 4: performing forward-backward difference processing on all the received echo signals within one time window in the slow-time dimension in the order of reception to obtain the plurality of differential signals, and respectively calculating a range spectrum for each of the plurality of differential signals to obtain a plurality of range spectra.

The joint processing operations described above differ in computational complexities and target detection capabilities. They may be systematically selected based on the application scenario and the radar's power consumption level, and this application does not impose restrictions on such selection.

In some embodiments, the radar is an FMCW radar or an SFCW radar, and the detection target (i.e., the desired target) of the radar is the first type of target. The radar transmits a fourth frame format detection signal every Δt₂ and receives corresponding echo signals. The fourth frame format detection signal of each frame is configured as a ranging signal waveform. The echo signals from the 1st to the Wth frame are considered as signals within the first time window, the echo signals from the (1+W)th to the (2W)th frame are considered as signals within the second time window, and so on. The W frames of echo signals within each time window form a data matrix. After performing static component suppression on the obtained data matrix, a range-velocity spectrum may be computed and generated. Target detection is then performed on the obtained range-velocity spectrum, and the detected target is filtered according to the predetermined range threshold and the predetermined velocity threshold. When the target still exists after filtering, it is determined that the first type of target exists; otherwise, it is determined that no target is found (the target has disappeared). Alternatively, the data with the same sampling point number in each data matrix may be averaged separately, and the range spectrum may be computed and generated according to the averaged data. The target is detected on the obtained range spectrum, and the detected target is filtered according to the predetermined range threshold. When the target still exists after filtering, it is determined that the first type of target is present; otherwise, it is determined that no target is found (the target has disappeared). The generation of the range-velocity spectra or the range spectra and the target detection on the range-velocity spectra or the range spectra are implemented by conventional technologies in the art, and this application does not impose restrictions on the calculation and detection details.

In some embodiments, the radar is a Doppler radar or an SFCW radar, and the detection target (i.e., the desired target) of the radar is the first type of target. The radar transmits the fourth frame format detection signal every Δt₂ and receives the corresponding echo signals. The fourth frame format detection signal of each frame is configured as the Doppler radar waveform. The echo signals from 1st frame to Wth frame serve as signals within the first time window, the echo signals from (1+W)th frame to (2W)th frame serve as signals within the second time window, and so on. The W frames of echo signals within each time window form a data vector. After suppressing the stationary components of the obtained data vector, the velocity spectrum is computed and generated. The target is detected on the obtained velocity spectrum, and the detected target is filtered according to a predetermined velocity threshold. When the target still exists after filtering, it is determined that the first type of target is present; otherwise, it is determined that no target is found (the target has disappeared). Herein, the generation of the velocity spectrum and target detection on the velocity spectrum are implemented by conventional techniques in the art, and this application does not impose restrictions on the calculation and detection details.

In the above two embodiments, the detected target in the fourth operating mode is the first type of target, meaning that the radar detects moving targets in the environment, including human movement and micro-movements, etc. Compared with the third operating mode, the radar has a lower algorithm detection load. On the premise that the third type of target has been detected in the third operating mode, the radar switches to the fourth operating mode for target detection at a low computational load, only determines whether the target has disappeared without performing precise parameter measurement and tracking of the target, which helps reduce computational power consumption of the radar.

In some embodiments, the radar is an FMCW radar or an SFCW radar, and the detection target (i.e., the desired target) of the radar is the second type of target. The radar transmits the fourth frame format detection signal every Δt₂ and receives the corresponding echo signals. The fourth frame format detection signal of each frame includes N ranging signal waveforms arranged at unequal time intervals. The arrangement of the N ranging signal waveforms in the time dimension is the same as that in the second operating mode. The time span of each time window is Δt₂. Within each time window, the radar receives the echo signal of one frame corresponding to the fourth frame format detection signal (i.e., the echo signal of the N ranging signal waveforms arranged at unequal time intervals). The radar processes the received echo signal in the same manner as in the embodiment of the second operating mode to detect whether the second type of target is present.

In some embodiments, the radar is a Doppler radar or an SFCW radar, and the detection target (i.e., the desired target) of the radar is the second type of target. The radar transmits the fourth frame format detection signal every Δt₂ and receives the corresponding echo signals. The fourth frame format detection signal of each frame includes N Doppler radar signal waveforms arranged at unequal time intervals. The arrangement of the N Doppler radar signal waveforms in the time dimension is the same as that in the second operating mode. The time span of each time window is Δt₂. Within each time window, the radar receives the echo signal of one frame corresponding to the fourth frame format detection signal (the echo signal of the N Doppler radar signal waveforms arranged at unequal time intervals). The radar processes the received echo signal in the same manner as in the embodiment of the second operating mode to detect whether the second type of target is present.

In the above two embodiments, the detected target in the fourth operating mode is the second type of target, meaning that the radar detects non-low-speed interfering targets in the environment. Compared with the third operating mode, the radar has a lower algorithm detection load. On the premise that the third type of target has been detected in the third operating mode, the radar switches to the fourth operating mode for target detection at a low computational load, only determining whether the target has disappeared without performing precise parameter measurement and tracking of the target, which helps reduce the computational power consumption of the radar.

In some embodiments, the radar is an FMCW radar or an SFCW radar, and the detection target (i.e., the desired target) of the radar is the third type of target (human movement). The radar transmits the fourth frame format detection signal every Δt₂ and receives the corresponding echo signals. The fourth frame format detection signal of each frame includes Q ranging signal waveforms arranged at equal time intervals. The time span of each time window is Δt₂. Within each time window, the radar receives the echo signal of one frame corresponding to the fourth frame format detection signal (the echo signal of the Q ranging signal waveforms arranged at equal time intervals). The radar processes the received echo signal in the same manner as in the embodiment of the third operating mode to detect whether the third type of target has disappeared.

In some embodiments, the radar is an FMCW radar or an SFCW radar, and the detection target (i.e., the desired target) of the radar is the third type of target (human vital signs). The radar transmits the fourth frame format detection signal every Δt₂ and receives the corresponding echo signals. The fourth frame format detection signal of each frame includes Q ranging signal waveforms arranged at equal time intervals. The time span of each time window is Δt₂. Within each time window, the radar receives the echo signal of one frame corresponding to the fourth frame format detection signal (i.e., the echo signal of the Q ranging signal waveforms arranged at equal time intervals). The radar processes the received echo signal in the same manner as in the embodiment of the third operating mode to detect whether the third type of target has disappeared.

In some embodiments, the radar is a Doppler radar or an SFCW radar, and the detection target (i.e., desired target) of the radar is the third type of target (i.e., human vital signs). The radar transmits the fourth frame format detection signal every Δt₂ and receives the corresponding echo signals. The fourth frame format detection signal of each frame contains Q Doppler radar signal waveforms arranged at equal time intervals. The time span of each time window is Δt₂. Within each time window, the radar receives the echo signal of one frame corresponding to the fourth frame format detection signal (i.e., the echo signal of the Q Doppler radar signal waveforms arranged at equal time intervals). The radar processes the received echo signal in the same manner as in the embodiment of the third operating mode to detect whether the third type of target has disappeared.

In the above three embodiments, the time interval Δt₂ may be set to be greater than Δt₀, meaning that after the third type of target has been detected in the third operating mode, the radar switches to the fourth operating mode to detect whether the third type of target has disappeared at a lower detection frame rate. At this time, compared with the third operating mode, the fourth operating mode can determine whether the third type of target has disappeared at lower power consumption.

In each embodiment of the fourth operating mode, optionally, when the radar supports angle measurement, angle measurement may be performed to obtain the angle information of the target detected in the embodiments, thereby further determining whether the detected target is the desired target in the fourth operating mode. Herein, the angle information includes the horizontal angle, azimuth angle, and elevation angle. In some embodiments of the above fourth operating mode, when the detected target is within a predetermined angle range (i.e., including a predetermined horizontal angle, a predetermined azimuth angle, and a predetermined elevation angle range), it is determined that the detected target is the desired target in the fourth operating mode; otherwise, the detected target is not the desired target in the fourth operating mode. In the embodiment of the fourth operating mode as described above, in one time window, when none of the detected targets is within the predetermined angle range, it is determined that no targets are found in this time window, that is, the targets have disappeared; otherwise, it is determined that the targets have not disappeared in this time window.

When detecting that the target has disappeared in the fourth operating mode, the radar sends an indication signal indicative of target disappearance to the main control unit, which then performs subsequent linked operations, for example sending instructions to a controlled device via a wired or a wireless connection to instruct the controlled device to perform a responsive action. This application imposes no limitation on such related implementations.

Furthermore, the above radar may further coexist with a passive infrared detector (PIR) to perform the radar target detection method described above. In some embodiments, when the passive infrared detector is operating, the radar remains powered-off or in a sleep state. When the passive infrared detector detects the target, the radar is powered on or exits the sleep state and enters the first operating mode, and the operating duration of the radar in the first operating mode is timed. The radar continues to operate according to the hierarchical operating mechanism from the first operating mode to the fourth operating mode as described above. When the operation duration of the radar in the first operating mode expires, the radar switches back to the powered-off or sleep state. When the radar is in the second operating mode and needs to exit this mode, the radar switches back to the powered-off or sleep state. When the radar does not detect a target and needs to exit the third operating mode while in the third operating mode, the radar switches back to the powered-off or sleep state. When the radar is in the fourth operating mode and needs to exit the fourth operating mode, the radar switches back to the powered-off or sleep state.

In some embodiments, when the passive infrared detector is operating, the radar remains powered-off or in the sleep state. When the passive infrared detector detects the target, the radar is powered on or exits the sleep state and enters the second operating mode. The radar continues to operate according to the hierarchical operating mechanism from the second operating mode to the fourth operating mode described above. When the radar is in the second operating mode and needs to exit the second operating mode, the radar switches back to the powered-off or sleep state. When the radar needs to exit the third operating mode due to not detecting a target while in the third operating mode, the radar switches back to the powered-off or sleep state. When the radar is in the fourth operating mode and needs to exit the fourth operating mode, the radar switches back to the powered-off or sleep state.

In some embodiments, when the passive infrared detector is operating, the radar remains powered-off or in the sleep state. When the passive infrared detector detects the target, the radar is powered on or exits the sleep state and enters the third operating mode. The radar continues to operate according to the hierarchical operating mechanism from the third operating mode to the fourth operating mode as described above. When the radar needs to exit the third operating mode due to not detecting a target while in the third operating mode, the radar switches back to the powered-off or sleep state. When the radar is in the fourth operating mode and needs to exit the fourth operating mode, the radar switches back to the powered-off or sleep state.

In some embodiments, when the passive infrared detector is operating, the radar remains powered-off or in the sleep state. When the passive infrared detector detects the target, the radar is powered on or exits the sleep state and enters the fourth operating mode. When the radar is in the fourth operating mode and needs to exit the fourth operating mode, the radar switches back to the powered-off or sleep state.

In the above four embodiments, selections should be made after systematic design based on factors such as the detection delay of the application scenario, power consumption allocation of multiple sensors, and anti-interference design of the radar. This application does not impose restrictions in this regard.

It should be noted that, according to the usage requirements of the application scenario, various embodiments for the same type of radar (i.e., FMCW radar, SFCW radar, or Doppler radar) under the four operating modes may be combined for use. For example, for an SFCW radar, the embodiment of the first operating mode (i.e., with the SFCW radar operating in a Doppler operating configuration), the embodiment of the second operating mode (i.e., with the SFCW radar operating in conventional ranging radar configuration), the embodiment of the third operating mode (i.e., with the SFCW radar operating in conventional ranging radar configuration), and the embodiment of the fourth operating mode (i.e., with the SFCW radar operating in Doppler radar configuration) may be combined for use.

It should be noted that, during actual implementation, it is recommended that, in the first operating mode, the radar detects and determines the presence of the first type of target from the range or velocity dimension. In the second operating mode, the radar jointly determines the presence of the second type of target from the range-Doppler dimension (after performing range filtering following Doppler-dimension low-velocity interference suppression). In the third operating mode, the radar detects and identifies the presence of the third type of target using multi-dimensional features (such as velocity, range, angle, micro-Doppler feature, kinematic trajectory, etc.). As the radar progresses from the first to the third operating mode, the complexity of radar signal processing gradually increases, and the judgment conditions for radar target detection gradually tighten. The set of the first type of target includes the second type of target, and the set of the second type of target includes the third type of target. Throughout the entire workflow, before determining the presence of the ultimately required third type of target, only partial detection functions are activated to confirm the presence of the first type of target and the second type of target. After excluding environmental interference, precise parameter measurement and tracking for the third type of target are initiated. This strategy reduces the radar's operating power consumption and conserves the radar's battery power.

As described above, the radar target detection method provided in the embodiments of this application may activate the first operating mode of the radar. When the first type of target is detected, the second operating mode of the radar is activated to detect the second type of target within a first predetermined period. When the second type of target is detected, the third operating mode of the radar is activated. Within a second predetermined period, the radar is controlled to extract the multi-dimensional measurement parameters to detect the third type of target. When the third type of target is detected within the second predetermined period, the fourth operating mode of the radar is activated, or the radar is controlled to remain in the third operating mode. In the fourth operating mode, the radar is controlled to divide time windows to perform target detection. If a desired target is detected within one time window, the radar is controlled to proceed to the next time window to continue detecting the presence of the desired target. The desired target includes the first type of target, the second type of target, and the third type of target, with the set of the first type of target including the second type of target and the set of the second type of target including the third type of target. The solution provided in the embodiments of this application may selectively activate the next operating mode of the radar based on the target detection results of the previous operating mode, gradually activating the radar's multi-dimensional parameter measurement function. This helps reduce the impact of various micro-motion interferences in interference environments and lower the radar's power consumption.

To implement the radar target detection method as described above, the embodiments of this application further provide a radar target detection apparatus, which may be specifically integrated into a terminal device such as a mobile phone, a tablet computer, or the like.

For example, as shown in FIG. 2, a structural schematic diagram of the radar target detection apparatus provided in the embodiments of this application is illustrated. The radar target detection apparatus may include:

A first detection unit 301, configured to activate a first operating mode of a radar, and when a first type of target is detected, activate a second operating mode of the radar;

A second detection unit 302, configured to detect a second type of target within a first predetermined period, and when the second type of target is detected within the first predetermined period, activate a third operating mode of the radar;

A third detection unit 303, configured to control the radar to extract multi-dimensional measurement parameters to detect a third type of target within a second predetermined period, and when the third type of target is detected within the second predetermined period, either activate a fourth operating mode of the radar or control the radar to remain in the third operating mode, wherein the multi-dimensional measurement parameters include range, velocity, angle, respiration and heartbeat rate, micro-Doppler feature, and motion trajectory; and

A fourth detection unit 304, configured to, in the fourth operating mode, control the radar to divide time windows to perform target detection, and when a desired target is detected within one time window, control the radar to proceed to a next time window to continue detecting the presence of the desired target, wherein the desired target includes the first type of target, the second type of target, and the third type of target, a set of the first type of target includes the second type of target, and a set of the second type of target includes the third type of target.

The radar target detection apparatus provided in the embodiments of this application may activate the first operating mode of the radar. When the first type of target is detected, it activates the second operating mode of the radar to detect the second type of target within a first predetermined period. When the second type of target is detected, it activates the third operating mode of the radar. Within a second predetermined period, the radar is controlled to extract multi-dimensional measurement parameters to detect the third type of target. When the third type of target is detected within the second predetermined period, it activates the fourth operating mode of the radar or controls the radar to remain in the third operating mode. In the fourth operating mode, the radar is controlled to divide time windows to perform target detection. If a desired target is detected within one time window, the radar is controlled to proceed to the next time window to continue to detect the presence of the desired target. The desired target includes the first type of target, the second type of target, and the third type of target, with the set of the first type of target including the second type of target and the set of the second type of target including the third type of target. The solution provided in the embodiments of this application may selectively activate the next operating mode of the radar based on the target detection results of the previous operating mode, gradually activating the radar's multi-dimensional parameter measurement function. This helps reduce the impact of various micro-motion interferences in interference environments and lower the radar's power consumption.

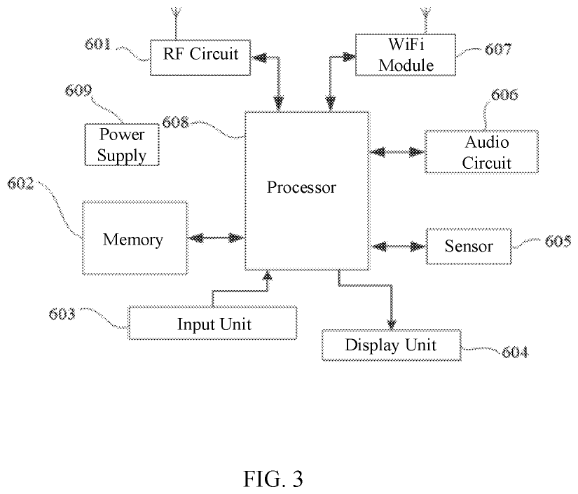

The embodiments of this application further provide a terminal. As shown in FIG. 3, the terminal may include components such as a radio frequency (RF) circuit 601, a memory 602 comprising one or more computer-readable storage media, an input unit 603, a display unit 604, a sensor 605, an audio circuit 606, a wireless fidelity (WiFi) module 607, a processor 608 comprising one or more processing cores, and a power supply 609. Those skilled in the art may appreciate that the terminal structure shown in FIG. 3 does not constitute a limitation on the terminal and may include more or fewer components than those illustrated, or some components may be combined, or different component arrangements may be used. Among them:

The RF circuit 601 is configured to receive and send signals during information transmission and reception, or phone calls. Specifically, after receiving downlink information from a base station, it hands over the downlink information to one or more processors 608 to be processed. Additionally, it sends uplink-related data to the base station. Typically, the RF circuit 601 includes, but is not limited to, an antenna, at least one amplifier, a tuner, one or more oscillators, a subscriber identity module (SIM) card, a transceiver, a coupler, a low noise amplifier (LNA), and a duplexer, and the like. Furthermore, the RF circuit 601 can also communicate with networks and other devices via wireless communication. Wireless communication can utilize any communication standard or protocol, including but not limited to Global System for Mobile Communication (GSM), General Packet Radio Service (GPRS), Code Division Multiple Access (CDMA), Wideband Code Division Multiple Access (WCDMA), Long Term Evolution (LTE), email, Short Messaging Service (SMS), etc.

The memory 602 is configured to store software programs and modules. By running the software programs and modules stored in the memory 602, the processor 608 is configured to execute various functional applications and information processing. The memory 602 mainly includes a program storage area and a data storage area. The program storage area can store an operating system and application programs required for at least one function (such as sound playback and image playback functions, etc.), and the like. The data storage area can store data created based on the usage of the terminal (such as audio data, a phonebook, etc.), and the like. Additionally, the memory 602 may include high-speed random access memory and may also include non-volatile memory, such as at least one disk storage device, a flash memory device, or other volatile solid-state storage devices. Correspondingly, the memory 602 can also include a memory controller to provide access to the memory 602 by the processor 608 and the input unit 603.

The input unit 603 is configured to receive input numeric or character information and generate keyboard, mouse, joystick, optical, or trackball signal inputs related to user settings and function control. Specifically, in a specific embodiment, the input unit 603 can include a touch-sensitive surface and other input devices. The touch-sensitive surface, also known as a touch display screen or touchpad, can collect touch operations performed by the user on or near it (e.g., operations performed by the user on or near the touch-sensitive surface using any suitable object or accessory, such as a finger or a stylus) and drive corresponding connecting devices based on a preset program. Optionally, the touch-sensitive surface can include two parts: a touch detection device and a touch controller. The touch detection device detects the user's touch position and detects signals brought by touch operations, transmitting the signals to the touch controller. The touch controller receives touch information from the touch detection device, converts the touch information into touch point coordinates, and sends the touch point coordinates to the processor 608. It can also receive commands from the processor 608 and execute them. Additionally, the touch-sensitive surface can be implemented using various types, such as resistive, capacitive, infrared, and surface acoustic wave. Besides the touch-sensitive surface, the input unit 603 can also include other input devices. Specifically, other input devices can include, but are not limited to, one or more of a physical keyboard, function keys (e.g., volume control, keyswitch keys, etc.), a trackball, a mouse, and a joystick, and the like.

The display unit 604 is configured to display information input by the user or information provided to the user, as well as various graphical user interfaces of the terminal. These graphical user interfaces can be composed of graphics, text, icons, videos, and any combination thereof. The display unit 604 may include a display panel. Optionally, the display panel can be configured in the form of a Liquid Crystal Display (LCD) or an Organic Light-Emitting Diode (OLED), or the like. Furthermore, a touch-sensitive surface may cover the display panel. When the touch-sensitive surface detects a touch operation on or near it, it transmits the information to the processor 608 to determine the type of touch event. Subsequently, the processor 608 provides corresponding visual output on the display panel based on the type of touch event. Although in FIG. 3, the touch-sensitive surface and the display panel are implemented as two separate components for input and output functions, in certain embodiments, the touch-sensitive surface and the display panel can be integrated to achieve both input and output functions.

The terminal may also include at least one sensor 605, such as an optical sensor, a motion sensor, and other sensors. Specifically, the optical sensor may include an ambient light sensor and a proximity sensor. The ambient light sensor can adjust the brightness of the display panel based on the intensity of ambient light, while the proximity sensor can turn off the display panel and/or backlight when the terminal is moved close to the ear. As a type of motion sensor, the gravity acceleration sensor can detect the magnitude of acceleration in various directions (typically three axes) and can detect the magnitude and direction of gravity when stationary. It can be used for applications that recognize the orientation of a terminal such as a mobile phone (e.g., landscape/portrait mode switching, related games, magnetometer attitude calibration) and vibration recognition functions (e.g., pedometer, tapping), and the like. Other sensors that the terminal may be equipped with, such as gyroscopes, barometers, hygrometers, thermometers, and infrared sensors, etc., will not be elaborated on here.

The audio circuit 606, speaker, and microphone may provide an audio interface between the user and the terminal. The audio circuit 606 can transmit electrical signals converted from received audio data to the speaker, which converts them into sound signals for output. On the other hand, the microphone converts collected sound signals into electrical signals, which are then received by the audio circuit 606 and converted into audio data. The audio data is further output to the processor 608 for processing, and is then transmitted via the RF circuit 601 to another terminal (for example), or output to the memory 602 for further processing. The audio circuit 606 may also include an earphone jack to provide communication between external headphones and the terminal.

WiFi is a short-range wireless transmission technology. The terminal, through the WiFi module 607, can assist users in sending and receiving emails, browsing web pages, and accessing streaming media, e.g., providing them with wireless broadband internet access. Although FIG. 3 shows the WiFi module 607, it should be understood that it is not an essential component of the terminal and can be omitted as needed without altering the essence of the application.

The processor 608 is the control center of the terminal, connecting various parts of the entire terminal through various interfaces and circuits. By running or executing software programs and/or modules stored in the memory 602 and calling data stored in the memory 602, the processor 608 performs various functions of the terminal and processes data, thereby providing overall monitoring of the terminal. Optionally, the processor 608 may include one or more processing cores. Preferably, the processor 608 may integrate an application processor and a modem processor, wherein the application processor mainly handles the operating system, user interface, and application programs, etc., while the modem processor mainly handles wireless communications. It should be understood that the modem processor may also not be integrated into the processor 608.

The terminal also includes a power supply 609 (such as a battery) that supplies power to various components. Preferably, the power supply 609 can be logically connected to the processor 608 through a power management system, thereby enabling functions such as charging, discharging, and power consumption management through the power management system. The power supply 609 may also include any one or more DC or AC power supplies, a recharging system, a power failure detection circuit, a power converter or inverter, a power status indicator, and other components.

Although not shown, the terminal may also include components such as a camera and a Bluetooth module, which will not be elaborated on here. Specifically, in this embodiment, the processor 608 in the terminal will load executable files corresponding to the processes of one or more application programs into the memory 602 according to the following instructions and run the application programs stored in the memory 602 to achieve various functions:

Activating a first operating mode of a radar, and when a first type of target is detected, activating a second operating mode of the radar;

Detecting a second type of target within a first predetermined period, and when the second type of target is detected within the first predetermined period, activating a third operating mode of the radar;

Controlling the radar to extract multi-dimensional measurement parameters to detect a third type of target within a second predetermined period, and when the third type of target is detected within the second predetermined period, either activating a fourth operating mode of the radar or controlling the radar to remain in the third operating mode, wherein the multi-dimensional measurement parameters include range, velocity, angle, respiration and heartbeat rate, micro-Doppler feature, and motion trajectory; and

In the fourth operating mode, controlling the radar to divide time windows to perform target detection, and when a desired target is detected within one time window, controlling the radar to proceed to a next time window to continue detecting the presence of the desired target, wherein the desired target includes the first type of target, the second type of target, and the third type of target, a set of the first type of target includes the second type of target, and a set of the second type of target includes the third type of target.

In the above embodiments, the descriptions of each embodiment focus on different aspects. For parts not elaborated in a certain embodiment, reference can be made to the detailed descriptions of the radar target detection method provided above, which will not be repeated here.

As described above, it can be seen that the terminal in the embodiments of this application can activate the first operating mode of the radar. When the first type of target is detected, it activates the second operating mode of the radar. Within the first predetermined period, it detects the second type of target. When the second type of target is detected, it activates the third operating mode of the radar. Within the second predetermined period, the radar is controlled to perform multi-dimensional measurement parameter extraction to detect the third type of target. When the third type of target is detected within the second predetermined period, it activates the fourth operating mode of the radar or controls the radar to remain in the third operating mode. In the fourth operating mode, the radar is controlled to divide time windows to perform target detection. If the desired target is detected within one time window, the radar is controlled to proceed to the next time window to continue detecting the presence of the desired target. The desired target includes the first, second, and third types of targets. The set of the first type of target includes the second type of target, and the set of the second type of target includes the third type of target. The solution provided in the embodiments of the application can selectively activate the next operating mode of the radar based on the target detection results of the previous operating mode, gradually activating the multi-dimensional parameter measurement function of the radar. This helps reduce the impact of various micro-motion interferences in interference environments and lower the power consumption of the radar.

Those skilled in the art can understand that all or some of the steps in the various methods described in the above embodiments can be completed by instructions or by controlling relevant hardware through instructions. The instructions may be stored in a computer-readable storage medium and loaded and executed by a processor.

Therefore, the embodiments of this application provide a computer-readable storage medium storing a plurality of instructions. The instructions may be loaded and executed by a processor to perform the steps in any of the radar target detection methods provided in the embodiments of this application. For example, the instructions may perform the following steps:

Activating a first operating mode of a radar, and when a first type of target is detected, activating a second operating mode of the radar;

Detecting a second type of target within a first predetermined period, and when the second type of target is detected within the first predetermined period, activating a third operating mode of the radar;

Controlling the radar to extract multi-dimensional measurement parameters to detect a third type of target within a second predetermined period, and when the third type of target is detected within the second predetermined period, either activating a fourth operating mode of the radar or controlling the radar to remain in the third operating mode, wherein the multi-dimensional measurement parameters include range, velocity, angle, respiration and heartbeat rate, micro-Doppler feature, and motion trajectory; and

In the fourth operating mode, controlling the radar to divide time windows to perform target detection, and when a desired target is detected within one time window, controlling the radar to proceed to a next time window to continue detecting the presence of the desired target, wherein the desired target includes the first type of target, the second type of target, and the third type of target, a set of the first type of target includes the second type of target, and a set of the second type of target includes the third type of target.

The specific implementations of the above operations can be found in the previous embodiments and will not be repeated here.

The computer-readable storage medium may include a Read Only Memory (ROM), a Random Access Memory (RAM), a magnetic disk, an optical disk, etc.

Since the instructions stored in the computer-readable storage medium can execute the steps in any of the radar target detection methods provided in the embodiments of this application, the beneficial effects that can be achieved by any of the radar target detection methods provided in the embodiments of this application can also be achieved. For details, refer to the previous embodiments, which will not be repeated here.

In the above, detailed introductions are provided for the radar target detection method, apparatus, terminal, and computer-readable storage medium according to the embodiments of this application. Specific examples have been used herein to illustrate the principles and implementations of this application. The above descriptions of the embodiments are only intended to help understand the method and its core ideas of this application. Meanwhile, for those skilled in the art, modifications may be made to the specific implementations and application scopes according to the ideas of this application. In summary, the content of this specification should not be construed as a limitation to this application.

1. A radar target detection method, comprising:
activating a first operating mode of a radar, and when a first type of target is detected, activating a second operating mode of the radar;
detecting a second type of target within a first predetermined period, and when the second type of target is detected within the first predetermined period, activating a third operating mode of the radar;
controlling the radar to extract multi-dimensional measurement parameters to detect a third type of target within a second predetermined period, and when the third type of target is detected within the second predetermined period, either activating a fourth operating mode of the radar or controlling the radar to remain in the third operating mode, wherein the multi-dimensional measurement parameters include range, velocity, angle, respiration and heartbeat rate, micro-Doppler feature, and motion trajectory; and
in the fourth operating mode, controlling the radar to divide time windows to perform target detection, and when a desired target is detected within one time window, controlling the radar to proceed to a next time window to continue detecting the presence of the desired target, wherein the desired target includes the first type of target, the second type of target, and the third type of target, a set of the first type of target includes the second type of target, and a set of the second type of target includes the third type of target.

2. The radar target detection method of claim 1, wherein after activating the first operating mode of the radar, the method further comprises:
periodically transmitting a first frame format detection signal and performing a first type of target detection based on a received signal;
when the radar is a ranging radar, generating a range spectrum based on the received signal of each frame and performing the first type of target detection on the range spectrum; and
when the radar is a Doppler radar, generating a velocity spectrum based on the received signal of each frame and performing the first type of target detection on the velocity spectrum.

3. The radar target detection method of claim 1, wherein after activating the second operating mode of the radar, the method further comprises:
transmitting a second frame format detection signal for a plurality of times, and weight-combining data with a same sampling point number among all received signals to obtain a combined signal, wherein transmitted second frame format detection signals are arranged at unequal time intervals;
when the radar is a ranging radar, generating a range spectrum based on the combined signal and performing a second type of target detection on the range spectrum; and
when the radar is a Doppler radar, computing an energy value of the combined signal and performing the second type of target detection based on a comparison result between the energy value and an energy threshold.

4. The radar target detection method of claim 1, wherein after activating the second operating mode of the radar, the method further comprises:
transmitting a second frame format detection signal for a plurality of times and performing forward-backward difference processing on all received signals in order of reception to obtain a plurality of differential signals, wherein transmitted second frame format detection signals are arranged at unequal time intervals;
when the radar is a ranging radar, generating a plurality of range spectra based on the plurality of differential signals and performing a second type of target detection based on a number of detected targets in the plurality of range spectra; and
when the radar is a Doppler radar, computing energy values of the plurality of differential signals and performing the second type of target detection based on a number of the energy values that exceed an energy threshold.

5. The radar target detection method of claim 1, wherein controlling the radar to extract the multi-dimensional measurement parameters comprises:
periodically transmitting a third frame format detection signal and receiving corresponding echo signals;
when the radar is a ranging radar, generating a range-velocity spectrum based on a plurality of received echo signals for a same frame and obtaining the multi-dimensional measurement parameters based on a plurality of range-velocity spectra; and
when the radar is a Doppler radar, generating a velocity spectrum based on a plurality of received echo signals for a same frame and obtaining the multi-dimensional measurement parameters based on a plurality of velocity spectra.

6. The radar target detection method of claim 1, wherein controlling the radar to divide the time windows to perform the target detection comprises:
performing joint processing on all echo signals received within a current time window to perform the target detection, wherein performing the joint processing is selected from one of:
performing a two-dimensional Fourier transform on all the echo signals to obtain a range-velocity spectrum;
performing arithmetic averaging on data with a same sampling point number among all the echo signals and computing a range spectrum based on arithmetically averaged data;
weight-combining the data with the same sampling point number among all the echo signals to obtain a combined signal and generating a range spectrum based on the combined signal; and
performing forward-backward difference processing on all the echo signals in order of reception to obtain a plurality of differential signals, and respectively calculating a range spectrum for each of the plurality of differential signals to obtain a plurality of range spectra.

7. The radar target detection method of claim 1, wherein after detecting the target in the second and fourth operating modes, the method further comprises:
obtaining angle information of a detected target, wherein the angle information includes a horizontal angle, an azimuth angle, and an elevation angle;
determining whether the angle information is within a predetermined range;
determining that the detected target is the desired target under a current operating mode when the angle information is within the predetermined range;
determining that the detected target is not the desired target under the current operating mode when the angle information is not within the predetermined range; and
wherein the desired target is the second type of target in the second operating mode, and the desired target includes the first type of target, the second type of target, and the third type of target in the fourth operating mode.

8. A terminal, comprising:
a memory, configured to store an application processing program; and
a processor, configured to execute the application processing program to perform a radar target detection method comprising:
activating a first operating mode of a radar, and when a first type of target is detected, activating a second operating mode of the radar;
detecting a second type of target within a first predetermined period, and when the second type of target is detected within the first predetermined period, activating a third operating mode of the radar;
controlling the radar to extract multi-dimensional measurement parameters to detect a third type of target within a second predetermined period, and when the third type of target is detected within the second predetermined period, either activating a fourth operating mode of the radar or controlling the radar to remain in the third operating mode, wherein the multi-dimensional measurement parameters include range, velocity, angle, respiration and heartbeat rate, micro-Doppler feature, and motion trajectory; and
in the fourth operating mode, controlling the radar to divide time windows to perform target detection, and when a desired target is detected within one time window, controlling the radar to proceed to a next time window to continue detecting the presence of the desired target, wherein the desired target includes the first type of target, the second type of target, and the third type of target, a set of the first type of target includes the second type of target, and a set of the second type of target includes the third type of target.

9. The terminal of claim 8, wherein after activating the first operating mode of the radar, the method further comprises:
periodically transmitting a first frame format detection signal and performing a first type of target detection based on a received signal;
when the radar is a ranging radar, generating a range spectrum based on the received signal of each frame and performing the first type of target detection on the range spectrum; and
when the radar is a Doppler radar, generating a velocity spectrum based on the received signal of each frame and performing the first type of target detection on the velocity spectrum.

10. The terminal of claim 8, wherein after activating the second operating mode of the radar, the method further comprises:
transmitting a second frame format detection signal for a plurality of times, and weight-combining data with a same sampling point number among all received signals to obtain a combined signal, wherein transmitted second frame format detection signals are arranged at unequal time intervals;
when the radar is a ranging radar, generating a range spectrum based on the combined signal and performing a second type of target detection on the range spectrum; and
when the radar is a Doppler radar, computing an energy value of the combined signal and performing the second type of target detection based on a comparison result between the energy value and an energy threshold.

11. The terminal of claim 8, wherein after activating the second operating mode of the radar, the method further comprises:
transmitting a second frame format detection signal for a plurality of times and performing forward-backward difference processing on all received signals in order of reception to obtain a plurality of differential signals, wherein transmitted second frame format detection signals are arranged at unequal time intervals;
when the radar is a ranging radar, generating a plurality of range spectra based on the plurality of differential signals and performing a second type of target detection based on a number of detected targets in the plurality of range spectra; and
when the radar is a Doppler radar, computing energy values of the plurality of differential signals and performing the second type of target detection based on a number of the energy values that exceed an energy threshold.

12. The terminal of claim 8, wherein controlling the radar to extract the multi-dimensional measurement parameters comprises:
periodically transmitting a third frame format detection signal and receiving corresponding echo signals;
when the radar is a ranging radar, generating a range-velocity spectrum based on a plurality of received echo signals for a same frame and obtaining the multi-dimensional measurement parameters based on a plurality of range-velocity spectra; and
when the radar is a Doppler radar, generating a velocity spectrum based on a plurality of received echo signals for a same frame and obtaining the multi-dimensional measurement parameters based on a plurality of velocity spectra.

13. The terminal of claim 8, wherein controlling the radar to divide the time windows to perform the target detection comprises:
performing joint processing on all echo signals received within a current time window to perform the target detection, wherein performing the joint processing is selected from one of:
performing a two-dimensional Fourier transform on all the echo signals to obtain a range-velocity spectrum;
performing arithmetic averaging on data with a same sampling point number among all the echo signals and computing a range spectrum based on arithmetically averaged data;
weight-combining the data with the same sampling point number among all the echo signals to obtain a combined signal and generating a range spectrum based on the combined signal; and
performing forward-backward difference processing on all the echo signals in order of reception to obtain a plurality of differential signals, and respectively calculating a range spectrum for each of the plurality of differential signals to obtain a plurality of range spectra.

14. The terminal of claim 8, wherein after detecting the target in the second and fourth operating modes, the method further comprises:
obtaining angle information of a detected target, wherein the angle information includes a horizontal angle, an azimuth angle, and an elevation angle;
determining whether the angle information is within a predetermined range;
determining that the detected target is the desired target under a current operating mode when the angle information is within the predetermined range;
determining that the detected target is not the desired target under the current operating mode when the angle information is not within the predetermined range; and
wherein the desired target is the second type of target in the second operating mode, and the desired target includes the first type of target, the second type of target, and the third type of target in the fourth operating mode.

15. A computer-readable storage medium, configured to store a plurality of instructions, wherein the plurality of instructions are configured to be executed by a processor to perform a radar target detection method comprising:
activating a first operating mode of a radar, and when a first type of target is detected, activating a second operating mode of the radar;
detecting a second type of target within a first predetermined period, and when the second type of target is detected within the first predetermined period, activating a third operating mode of the radar;
controlling the radar to extract multi-dimensional measurement parameters to detect a third type of target within a second predetermined period, and when the third type of target is detected within the second predetermined period, either activating a fourth operating mode of the radar or controlling the radar to remain in the third operating mode, wherein the multi-dimensional measurement parameters include range, velocity, angle, respiration and heartbeat rate, micro-Doppler feature, and motion trajectory; and
in the fourth operating mode, controlling the radar to divide time windows to perform target detection, and when a desired target is detected within one time window, controlling the radar to proceed to a next time window to continue detecting the presence of the desired target, wherein the desired target includes the first type of target, the second type of target, and the third type of target, a set of the first type of target includes the second type of target, and a set of the second type of target includes the third type of target.

16. The computer-readable storage medium of claim 15, wherein after activating the first operating mode of the radar, the method further comprises:
periodically transmitting a first frame format detection signal and performing a first type of target detection based on a received signal;
when the radar is a ranging radar, generating a range spectrum based on the received signal of each frame and performing the first type of target detection on the range spectrum; and
when the radar is a Doppler radar, generating a velocity spectrum based on the received signal of each frame and performing the first type of target detection on the velocity spectrum.

17. The computer-readable storage medium of claim 15, wherein after activating the second operating mode of the radar, the method further comprises:
transmitting a second frame format detection signal for a plurality of times, and weight-combining data with a same sampling point number among all received signals to obtain a combined signal, wherein transmitted second frame format detection signals are arranged at unequal time intervals;
when the radar is a ranging radar, generating a range spectrum based on the combined signal and performing a second type of target detection on the range spectrum; and
when the radar is a Doppler radar, computing an energy value of the combined signal and performing the second type of target detection based on a comparison result between the energy value and an energy threshold.

18. The computer-readable storage medium of claim 15, wherein after activating the second operating mode of the radar, the method further comprises:
transmitting a second frame format detection signal for a plurality of times and performing forward-backward difference processing on all received signals in order of reception to obtain a plurality of differential signals, wherein transmitted second frame format detection signals are arranged at unequal time intervals;
when the radar is a ranging radar, generating a plurality of range spectra based on the plurality of differential signals and performing a second type of target detection based on a number of detected targets in the plurality of range spectra; and
when the radar is a Doppler radar, computing energy values of the plurality of differential signals and performing the second type of target detection based on a number of the energy values that exceed an energy threshold.

19. The computer-readable storage medium of claim 15, wherein controlling the radar to extract multi-dimensional measurement parameters comprises:
periodically transmitting a third frame format detection signal and receiving corresponding echo signals;
when the radar is a ranging radar, generating a range-velocity spectrum based on a plurality of received echo signals for a same frame and obtaining the multi-dimensional measurement parameters based on a plurality of range-velocity spectra; and
when the radar is a Doppler radar, generating a velocity spectrum based on a plurality of received echo signals for a same frame and obtaining the multi-dimensional measurement parameters based on a plurality of velocity spectra.

20. The computer-readable storage medium of claim 15, wherein controlling the radar to divide the time windows to perform the target detection comprises:
performing joint processing on all echo signals received within a current time window to perform the target detection, wherein performing the joint processing is selected from one of:
performing a two-dimensional Fourier transform on all the echo signals to obtain a range-velocity spectrum;
performing arithmetic averaging on data with a same sampling point number among all the echo signals and computing a range spectrum based on arithmetically averaged data;
weight-combining the data with the same sampling point number among all the echo signals to obtain a combined signal and generating a range spectrum based on the combined signal; and
performing forward-backward difference processing on all the echo signals in order of reception to obtain a plurality of differential signals, and respectively calculating a range spectrum for each of the plurality of differential signals to obtain a plurality of range spectra.
